(19) United States
(12) Patent Application Publication
(10) Pub. No.: US 2024/0122500 A1
(43) Pub. Date: Apr. 18, 2024

(54) EAR-WEARABLE DEVICES FOR GAIT AND IMPACT TRACKING OF KNEE AND HIP REPLACEMENTS

(71) Applicant: Starkey Laboratories, Inc., Eden Prairie, MN (US)

(72) Inventors: David Alan Fabry, Eden Prairie, MN (US); Archelle Georgiou Feldshon, Wayzata, MN (US); Kyle Olson, St. Louis Park, MN (US); Paul Shriner, Hopkins, MN (US)

(21) Appl. No.: 18/373,629

(22) Filed: Sep. 27, 2023

Related U.S. Application Data
(60) Provisional application No. 63/411,991, filed on Sep. 30, 2022.

(51) Int. Cl. A61B 5/11; A61B 5/00

(52) U.S. Cl. CPC A61B 5/112; A61B 5/0022; A61B 5/1116; A61B 5/6815

(57) ABSTRACT

Embodiments herein relate to ear-wearable devices and systems with features to track orthopedic issues and orthopedic procedure recovery. In an embodiment, an ear-wearable device is included having a control circuit, a microphone, and a sensor package. The ear-wearable device can be configured to receive an input regarding an occurrence of an orthopedic procedure, evaluate signals from the sensor package and/or the microphone, and determine a rehabilitative status of a device wearer with respect to the orthopedic procedure. Other embodiments are also included herein.

This application claims the benefit of U.S. Provisional Application No. 63/411,991, filed Sep. 30, 2022, the content of which is herein incorporated by reference in its entirety.

### FIELD

Embodiments herein relate to ear-wearable devices. More specifically, embodiments herein relate to ear-wearable devices and system with features to track orthopedic issues and orthopedic procedure recovery.

### BACKGROUND

It is estimated that over 12 million adults see a physician each year in the United States about joint pain. Treatment options for joint pain can include physical therapy, physical activity modifications, nutrition modifications and support, braces and other devices, application of heat or ice, medications of many types (including pain medications, anti-inflammatory medications, and antirheumatic drugs, etc.), acupuncture, and various surgical procedures. Joint replacement operations have now become quite common. Common joints replaced include hips and knees, but other joints are also replaced including shoulders, ankles, elbows, wrists and the like. Joint replacement procedures can take several months post-operation to properly recover from.

### SUMMARY

Embodiments herein relate to ear-wearable devices and system with features to track orthopedic issues and orthopedic procedure recovery. In a first aspect, an ear-wearable device is included having a control circuit, a microphone, and a sensor package. The ear-wearable device can be configured to receive an input regarding an occurrence of an orthopedic procedure, evaluate signals from the sensor package and/or the microphone, and determine a rehabilitative status of a device wearer with respect to the orthopedic procedure.

In a second aspect, in addition to one or more of the preceding or following aspects, or in the alternative to some aspects, the orthopedic procedure can include a lower extremity orthopedic surgery. The lower extremity orthopedic surgery can include at least one selected from the group consisting of a hip replacement surgery, a knee replacement surgery, an ankle surgery, and a foot surgery.

In a third aspect, in addition to one or more of the preceding or following aspects, or in the alternative to some aspects, the orthopedic procedure can include treatment of a lower extremity bone fracture.

In a fourth aspect, in addition to one or more of the preceding or following aspects, or in the alternative to some aspects, the orthopedic procedure can include treatment of a hip fracture.

In a fifth aspect, in addition to one or more of the preceding or following aspects, or in the alternative to some aspects, the ear-wearable device can be configured to evaluate signals from the sensor package and/or the microphone for indicia of pain.

In a sixth aspect, in addition to one or more of the preceding or following aspects, or in the alternative to some aspects, the ear-wearable device can be configured to determine a post-procedural mobility.

In a seventh aspect, in addition to one or more of the preceding or following aspects, or in the alternative to some aspects, the ear-wearable device can be configured to determine a post-procedural mobility trend.

In an eighth aspect, in addition to one or more of the preceding or following aspects, or in the alternative to some aspects, the ear-wearable device can be configured to determine a post-procedural mobility value and compare the same with a pre-procedure mobility value.

In a ninth aspect, in addition to one or more of the preceding or following aspects, or in the alternative to some aspects, the ear-wearable device can be configured to evaluate a post-procedural gait parameter of the device wearer.

In a tenth aspect, in addition to one or more of the preceding or following aspects, or in the alternative to some aspects, the gait parameter can include at least one selected from the group consisting of gait velocity, step length, swing and stance phase, double support time, ground reaction forces, impulse, and propulsion during habitual walking.

In an eleventh aspect, in addition to one or more of the preceding or following aspects, or in the alternative to some aspects, the ear-wearable device can be configured to detect leg swinging associated with leg stiffness.

In a twelfth aspect, in addition to one or more of the preceding or following aspects, or in the alternative to some aspects, the ear-wearable device can be configured to evaluate a gait parameter symmetry of the device wearer.

In a thirteenth aspect, in addition to one or more of the preceding or following aspects, or in the alternative to some aspects, the ear-wearable device can be configured to evaluate step pace symmetry of the device wearer.

In a fourteenth aspect, in addition to one or more of the preceding or following aspects, or in the alternative to some aspects, the ear-wearable device can be configured to detect leaning of the device wearer during ambulation.

In a fifteenth aspect, in addition to one or more of the preceding or following aspects, or in the alternative to some aspects, the ear-wearable device can be configured to detect stumbling or wobbling in a particular direction.

In a sixteenth aspect, in addition to one or more of the preceding or following aspects, or in the alternative to some aspects, the ear-wearable device can be configured to detect an asymmetrical leaning pattern of the device wearer during ambulation.

In a seventeenth aspect, in addition to one or more of the preceding or following aspects, or in the alternative to some aspects, the ear-wearable device can be configured to evaluate signals from the microphone to detect one or more phrases or utterances indicative of pain, stiffness, or other discomfort associated with the orthopedic procedure.

In an eighteenth aspect, in addition to one or more of the preceding or following aspects, or in the alternative to some aspects, the ear-wearable device can be configured to detect a posture change or activity of the device wearer.

In a nineteenth aspect, in addition to one or more of the preceding or following aspects, or in the alternative to some aspects, the ear-wearable device can be configured to change a pain, stiffness, or other discomfort detection threshold value when the device wearer is detected to be sitting down, standing up, using stairs, or exercising.

In a twentieth aspect, in addition to one or more of the preceding or following aspects, or in the alternative to some aspects, the ear-wearable device can be configured to detect a posture change or activity of the device wearer and detect a left-right weight distribution during the posture change or activity.

In a twenty-first aspect, in addition to one or more of the preceding or following aspects, or in the alternative to some aspects, the ear-wearable device can be configured to query the device wearer regarding pain, stiffness, or other discomfort.

In a twenty-second aspect, in addition to one or more of the preceding or following aspects, or in the alternative to some aspects, the ear-wearable device can be configured to record detected indicia of pain, stiffness, or other discomfort experienced by the device wearer.

In a twenty-third aspect, in addition to one or more of the preceding or following aspects, or in the alternative to some aspects, the ear-wearable device can be configured to evaluate signals from the sensor package to detect possible post-procedural infection.

In a twenty-fourth aspect, in addition to one or more of the preceding or following aspects, or in the alternative to some aspects, the ear-wearable device can be configured to send an alert to a care provider if a possible post-procedural infection can be detected.

In a twenty-fifth aspect, in addition to one or more of the preceding or following aspects, or in the alternative to some aspects, the input can include an indicator of left vs. right procedures.

In a twenty-sixth aspect, in addition to one or more of the preceding or following aspects, or in the alternative to some aspects, the input can include an anatomical site of the procedure.

In a twenty-seventh aspect, in addition to one or more of the preceding or following aspects, or in the alternative to some aspects, the ear-wearable device can be configured to provide information to a care provider regarding a rehabilitative status of the device wearer.

In a twenty-eighth aspect, in addition to one or more of the preceding or following aspects, or in the alternative to some aspects, the ear-wearable device can be configured to provide a reminder to the device wearer if a detected post-procedural activity level crosses a threshold value.

In a twenty-ninth aspect, in addition to one or more of the preceding or following aspects, or in the alternative to some aspects, the ear-wearable device can be configured to provide a reminder to the device wearer if a duration of a detected post-procedural activity crosses a threshold value.

In a thirtieth aspect, in addition to one or more of the preceding or following aspects, or in the alternative to some aspects, the ear-wearable device can be configured to provide a reminder to the device wearer if a post-procedural activity that is not recommended can be detected.

In a thirty-first aspect, in addition to one or more of the preceding or following aspects, or in the alternative to some aspects, the ear-wearable device can be configured to provide reminders to the device wearer regarding recommended post-procedural activities.

In a thirty-second aspect, in addition to one or more of the preceding or following aspects, or in the alternative to some aspects, the recommended post-procedural activities can include stretching and/or exercises.

In a thirty-third aspect, in addition to one or more of the preceding or following aspects, or in the alternative to some aspects, the ear-wearable device can be configured to provide instructions to the device wearer regarding recommended post-procedural activities.

In a thirty-fourth aspect, a method of monitoring an individual after an orthopedic procedure can be included. The method can include receiving an input regarding an occurrence of the orthopedic procedure, evaluating signals from a sensor package and/or a microphone, and determining a rehabilitative status of a device wearer with respect to the orthopedic procedure.

In a thirty-fifth aspect, in addition to one or more of the preceding or following aspects, or in the alternative to some aspects, the method can further include evaluating signals from the sensor package and/or the microphone for indicia of pain.

In a thirty-sixth aspect, in addition to one or more of the preceding or following aspects, or in the alternative to some aspects, the method can further include determining a post-procedural mobility value.

In a thirty-seventh aspect, in addition to one or more of the preceding or following aspects, or in the alternative to some aspects, the method can further include determining a post-procedural mobility trend.

In a thirty-eighth aspect, in addition to one or more of the preceding or following aspects, or in the alternative to some aspects, the method can further include determining a post-procedural mobility value and compare the same with a pre-procedure mobility value.

In a thirty-ninth aspect, in addition to one or more of the preceding or following aspects, or in the alternative to some aspects, the method can further include evaluating a post-procedural gait parameter of the device wearer.

In a fortieth aspect, in addition to one or more of the preceding or following aspects, or in the alternative to some aspects, the method can further include detecting leg swinging associated with leg stiffness.

In a forty-first aspect, in addition to one or more of the preceding or following aspects, or in the alternative to some aspects, the method can further include evaluating a gait parameter symmetry of the device wearer.

In a forty-second aspect, in addition to one or more of the preceding or following aspects, or in the alternative to some aspects, the method can further include evaluating step pace symmetry of the device wearer.

In a forty-third aspect, in addition to one or more of the preceding or following aspects, or in the alternative to some aspects, the method can further include detecting leaning of the device wearer during ambulation.

In a forty-fourth aspect, in addition to one or more of the preceding or following aspects, or in the alternative to some aspects, the method can further include detecting stumbling or wobbling in a particular direction.

In a forty-fifth aspect, in addition to one or more of the preceding or following aspects, or in the alternative to some aspects, the method can further include detecting an asymmetrical leaning pattern of the device wearer during ambulation.

In a forty-sixth aspect, in addition to one or more of the preceding or following aspects, or in the alternative to some aspects, the method can further include evaluating signals from the microphone to detect one or more phrases or utterances indicative of pain, stiffness, or other discomfort associated with the orthopedic procedure.

In a forty-seventh aspect, in addition to one or more of the preceding or following aspects, or in the alternative to some aspects, the method can further include detecting a posture change or activity of the device wearer.

In a forty-eighth aspect, in addition to one or more of the preceding or following aspects, or in the alternative to some aspects, the method can further include changing a pain, stiffness, or other discomfort detection threshold value when the device wearer can be detected to be sitting down, standing up, using stairs, or exercising.

In a forty-ninth aspect, in addition to one or more of the preceding or following aspects, or in the alternative to some aspects, the method can further include detecting a posture change or activity of the device wearer and detecting a left-right weight distribution during the posture change or activity.

In a fiftieth aspect, in addition to one or more of the preceding or following aspects, or in the alternative to some aspects, the method can further include querying the device wearer regarding pain, stiffness, or other discomfort.

In a fifty-first aspect, in addition to one or more of the preceding or following aspects, or in the alternative to some aspects, the method can further include recording detected indicia of pain, stiffness, or other discomfort experienced by the device wearer.

In a fifty-second aspect, in addition to one or more of the preceding or following aspects, or in the alternative to some aspects, the method can further include evaluating signals from the sensor package to detect possible post-procedural infection.

In a fifty-third aspect, in addition to one or more of the preceding or following aspects, or in the alternative to some aspects, the method can further include sending an alert to a care provider if a possible post-procedural infection can be detected.

In a fifty-fourth aspect, in addition to one or more of the preceding or following aspects, or in the alternative to some aspects, the method can further include providing information to a care provider regarding a rehabilitative status of the device wearer.

In a fifty-fifth aspect, in addition to one or more of the preceding or following aspects, or in the alternative to some aspects, the method can further include providing a reminder to the device wearer if a detected post-procedural activity level crosses a threshold value.

In a fifty-sixth aspect, in addition to one or more of the preceding or following aspects, or in the alternative to some aspects, the method can further include providing reminders to the device wearer regarding recommended post-procedural activities.

In a fifty-seventh aspect, in addition to one or more of the preceding or following aspects, or in the alternative to some aspects, the method can further include providing instructions to the device wearer regarding recommended post-procedural activities.

In a fifty-eighth aspect, in addition to one or more of the preceding or following aspects, or in the alternative to some aspects, the method can further include providing a reminder to the device wearer if a detected post-procedural activity level crosses a threshold value.

In a fifty-ninth aspect, in addition to one or more of the preceding or following aspects, or in the alternative to some aspects, the method can further include providing a reminder to the device wearer if a duration of a detected post-procedural activity has crossed a threshold value.

In a sixtieth aspect, an ear-wearable device can be included having a control circuit, a microphone, and a sensor package. The sensor package can be in electrical communication with the control circuit. The ear-wearable device can be configured to evaluate signals from the sensor package and/or the microphone and determine an orthopedic condition status of a device wearer.

In a sixty-first aspect, in addition to one or more of the preceding or following aspects, or in the alternative to some aspects, the ear-wearable device can be configured to receive an input to initiate the orthopedic condition status tracking mode.

In a sixty-second aspect, in addition to one or more of the preceding or following aspects, or in the alternative to some aspects, the orthopedic condition can include a lower extremity joint condition.

In a sixty-third aspect, in addition to one or more of the preceding or following aspects, or in the alternative to some aspects, the ear-wearable device can be configured to evaluate signals from the sensor package and/or the microphone for indicia of pain.

In a sixty-fourth aspect, in addition to one or more of the preceding or following aspects, or in the alternative to some aspects, the ear-wearable device can be configured to determine a mobility baseline value.

In a sixty-fifth aspect, in addition to one or more of the preceding or following aspects, or in the alternative to some aspects, the ear-wearable device can be configured to determine a mobility trend.

In a sixty-sixth aspect, in addition to one or more of the preceding or following aspects, or in the alternative to some aspects, the ear-wearable device can be configured to evaluate a gait parameter of the device wearer.

In a sixty-seventh aspect, in addition to one or more of the preceding or following aspects, or in the alternative to some aspects, the gait parameter can include at least one selected from the group consisting of gait velocity, step length, swing and stance phase, double support time, ground reaction forces, impulse, and propulsion during habitual walking.

In a sixty-eighth aspect, in addition to one or more of the preceding or following aspects, or in the alternative to some aspects, the ear-wearable device can be configured to detect leg swinging associated with leg stiffness.

In a sixty-ninth aspect, in addition to one or more of the preceding or following aspects, or in the alternative to some aspects, the ear-wearable device can be configured to evaluate a gait parameter symmetry of the device wearer.

In a seventieth aspect, in addition to one or more of the preceding or following aspects, or in the alternative to some aspects, the ear-wearable device can be configured to evaluate step pace symmetry of the device wearer.

In a seventy-first aspect, in addition to one or more of the preceding or following aspects, or in the alternative to some aspects, the ear-wearable device can be configured to detect leaning of the device wearer during ambulation.

In a seventy-second aspect, in addition to one or more of the preceding or following aspects, or in the alternative to some aspects, the ear-wearable device can be configured to detect stumbling or wobbling in a particular direction.

In a seventy-third aspect, in addition to one or more of the preceding or following aspects, or in the alternative to some aspects, the ear-wearable device can be configured to detect an asymmetrical leaning pattern of the device wearer during ambulation.

In a seventy-fourth aspect, in addition to one or more of the preceding or following aspects, or in the alternative to some aspects, the ear-wearable device can be configured to evaluate signals from the microphone to detect one or more phrases or utterances indicative of pain, stiffness, or other discomfort associated with an orthopedic procedure.

In a seventy-fifth aspect, in addition to one or more of the preceding or following aspects, or in the alternative to some aspects, the ear-wearable device can be configured to detect a posture change or activity of the device wearer.

In a seventy-sixth aspect, in addition to one or more of the preceding or following aspects, or in the alternative to some aspects, the ear-wearable device can be configured to change a pain, stiffness, or other discomfort detection threshold value when the device wearer is detected to be sitting down, standing up, using stairs, or exercising.

In a seventy-seventh aspect, in addition to one or more of the preceding or following aspects, or in the alternative to some aspects, the ear-wearable device can be configured to detect a posture change or activity of the device wearer and detect a left-right weight distribution during the posture change or activity.

In a seventy-eighth aspect, in addition to one or more of the preceding or following aspects, or in the alternative to some aspects, the ear-wearable device can be configured to query the device wearer regarding pain, stiffness, or other discomfort.

In a seventy-ninth aspect, in addition to one or more of the preceding or following aspects, or in the alternative to some aspects, the ear-wearable device can be configured to record detected indicia of pain, stiffness, or other discomfort experienced by the device wearer.

In an eightieth aspect, in addition to one or more of the preceding or following aspects, or in the alternative to some aspects, the ear-wearable device can be configured to provide information to a care provider regarding the orthopedic condition status of the device wearer.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE FIGURES

Aspects may be more completely understood in connection with the following figures (FIGS.), in which:

FIG. 1 is a schematic view of a device wearer in accordance with various embodiments herein.

FIG. 2 is a schematic view of post-procedural recovery phases in accordance with various embodiments herein.

FIG. 3 is a schematic view of an ear-wearable device in accordance with various embodiments herein.

FIG. 4 is a schematic view of an ear-wearable device in an ear in accordance with various embodiments herein.

FIG. 5 is a schematic view of a device wearer in accordance with various embodiments herein.

FIG. 6 is a flow chart of operations of an exemplary embodiment herein.

FIG. 7 is a schematic view of gait aspects in accordance with various embodiments herein.

FIG. 8 is a schematic view of left and right steps in accordance with various embodiments herein.

FIG. 9 is a schematic view of a device wearer rising from a chair in accordance with various embodiments herein.

FIG. 10 is a schematic view of a device wearer in accordance with various embodiments herein.

FIG. 11 is a schematic view of an accessory device in accordance with various embodiments herein.

FIG. 12 is a schematic view of an accessory device in accordance with various embodiments herein.

FIG. 13 is a schematic block diagram of components of an ear-wearable device in accordance with various embodiments herein.

FIG. 14 is a schematic view of various components of a system in accordance with various embodiments herein.

While embodiments are susceptible to various modifications and alternative forms, specifics thereof have been shown by way of example and drawings, and will be described in detail. It should be understood, however, that the scope herein is not limited to the particular aspects described. On the contrary, the intention is to cover modifications, equivalents, and alternatives falling within the spirit and scope herein.

### DETAILED DESCRIPTION

As described above, joint replacement operations have now become quite common. However, joint replacement procedures can take several months post-operation to properly recover from. It is important that any issues arising during recovery are quickly identified. It is also important that patients follow care instructions including undertaking recommended rehabilitation activities as well as refraining from certain activities such as those that might place too much stress on the joint. Beyond joint replacements, similar considerations apply to other types of orthopedic procedures.

Embodiments herein can help to track recovery from joint replacements and other types of orthopedic procedures. For example, embodiments herein can help to track post-procedural activity and use of joints by looking at aspects such as how a device wearer is walking, standing, and sitting. The data gathered by the system can be used in various ways including, for example, being remotely reviewed by a clinician or care provider to help determine how the recovery is going and what, if any, changes may need to be made. As an example, an ear-wearable device herein can include a control circuit, a microphone, and a sensor package. The ear-wearable device can be configured to receive an input regarding an occurrence of an orthopedic procedure, evaluate signals from the sensor package and/or the microphone, and determine a rehabilitative status of a device wearer with respect to the orthopedic procedure.

Systems herein can monitor orthopedic condition and/or recovery status with respect to many different kinds of orthopedic issues. For example, orthopedic issues herein can include lower extremity orthopedic surgeries, procedures, and injuries. Exemplary lower extremity orthopedic surgeries can include, but are not limited to, hip replacement surgery, knee replacement surgery, ankle surgery, foot surgery and the like. In various embodiments, an orthopedic procedure herein can include treatment of a lower extremity bone fracture. In various embodiments, an orthopedic procedure herein can include treatment of a hip fracture.

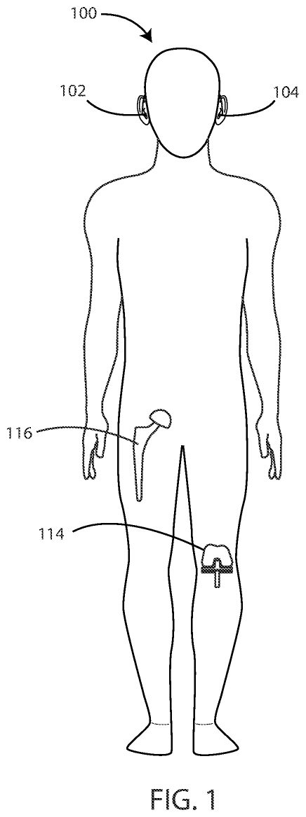

Referring now to FIG. 1, a schematic view is shown of a device wearer 100 with ear-wearable devices 102 and 104. However, in some cases, a device wearer 100 may only have a single ear-wearable device. The ear-wearable devices 102 and 104 can detect aspects of the health status of a device wearer and, specifically, aspects related to the post-operative recovery from a joint replacement. In this example, the device wearer 100 has an implanted knee replacement device 114. However, as referenced above, embodiments herein are applicable to other joint replacements as well. For example, the device wearer 100 may alternatively, or in addition, have an implanted hip replacement device 116.

In various embodiments, the ear-wearable devices 102, 104 can each include various components such as a control circuit, a microphone, and a sensor package. The ear-wearable devices 102, 104 can be configured to analyze data from the sensor package and/or the microphone to determine activity and/or physiological parameters of the device wearer 100. The ear-wearable devices 102, 104 can specifically be configured to evaluate data from the sensor package and/or the microphone to detect aspects related to the post-operative recovery from a joint replacement.

In various embodiments, ear-wearable devices herein can be configured to provide information related to post-operative recovery of the device wearer to various third parties such as care providers and/or clinicians. In some embodiments, the ear-wearable device can be configured to provide information to a care provider regarding the orthopedic condition status of the device wearer. In this manner, such third parties can be kept informed of the status of the device wearer and/or the recovery process. In some cases, the information provided can be routine health information relevant to post-operative recovery and can be reported in real time or according to a fixed schedule. However, in some embodiments, the wearable device can provide an alert, notification or warning if an issue is detected. The alert can be provided to the device wearer and/or to one or more third parties such as a care provider, clinician, or other responsible party. In some cases, the alert may take the form of an electronic data transmission. In some embodiments, an alert can be a video and/or an audio alert using components of the ear-wearable device such as an electroacoustic transducer or using components of another device.

In various scenarios, embodiments here can also use different and/or multiple types of data to improve the accuracy of monitoring and/or detection of events. Accuracy can be important as a device that issues too many false alerts or warnings will soon be ignored. For example, devices herein can use data regarding one or more of an activity level of a device wearer, movement data, geolocation data, microphone data, data from separate devices, medical record data, food and/or water intake data, medication intake data, and the like to improve the accuracy of monitoring and/or event detection herein. In some cases, data used can originate with sensors of the device and/or system. In some cases, the data can originate with sensors of other devices or components can be transmitted to the devices and/or systems herein. In still other cases, the data may not be sensor data, but another type of data that may impact accurate monitoring and/or detection of events, such as other health data regarding the device wearer.

In various embodiments, ear-wearable devices herein can be configured to receive an input regarding an occurrence of an orthopedic procedure or an orthopedic issue. For example, an input could be provided and received through a microphone, though button presses, through a wireless communication, or the like. The input could be provided by an individual or by a system. In some cases, a clinician may provide an input related to an orthopedic procedure. In some cases, a medical records system or patient management system may provide an input or query response related to an orthopedic procedure. In various embodiments, the input can include an anatomical site of the procedure. It can be important to know what side of the body the procedure or issue has impacted. In various embodiments, the input can include an indicator of left vs. right procedures.

In various embodiments, ear-wearable devices herein can be configured to automatically detect an occurrence of an orthopedic procedure or orthopedic issue. For example, signals from sensors herein can be analyzed to detect a pattern indicating that an orthopedic procedure has been performed and/or that an orthopedic issue exists. In some cases, data can be matched using pattern matching algorithms against templates for specific orthopedic procedures or issues (such as a left-side hip replacement template, a right-side hip replacement template, a left-side knee replacement template, a right-side knee replacement template, and the like). If a match is found against a particular template, then the system can determine that the device wearer has had an orthopedic procedure or issue associated with the matching template. Details regarding exemplary pattern matching techniques are provided below.

In some embodiments, whether receiving an input that an orthopedic procedure has been performed, that an orthopedic issue exists, or automatically determining the same, ear-wearable devices herein can enter an operation mode wherein monitoring and/or detection is performed with respect to orthopedic aspects as described herein such as an orthopedic condition status tracking mode. For example, in various embodiments, the ear-wearable device can be configured to evaluate signals from a sensor package and/or a microphone in such a tracking mode. In various embodiments, the ear-wearable device can be configured to determine a rehabilitative status of a device wearer with respect to an orthopedic procedure in such a tracking mode. In various embodiments, the ear-wearable device can be configured to provide information to a care provider regarding a rehabilitative status of the device wearer in such a tracking mode. In some embodiments, detected activity levels and/or mobility values can be used as a proxy for rehabilitative status. In some embodiments, detected activity levels and/or mobility values in combination with one or more other data types discussed herein can be used as a proxy for rehabilitative status.

In various embodiments, ear-wearable devices herein can be configured to evaluate signals from a sensor package and/or a microphone for indicia of pain and/or severity of the same or other discomfort. While some pain and discomfort can be normal during the recovery phase after a joint replacement or other orthopedic procedure, pain and significant discomfort can also be a marker of a problem or developing issue in the recovery process. Pain and significant discomfort can also be a marker of an orthopedic issue. As such, embodiments herein can evaluate data to detect and/or infer the occurrence of pain and/or discomfort and, in some embodiments, a degree of the same. By way of example, in some embodiments, the system herein can use microphone data to detect occurrences of sounds (e.g., “aaaahhhh”) and/or uttered words (e.g., “ouch”) consistent with pain and/or discomfort. In some embodiments, the system can query the device wearer periodically to detect the occurrence and/or severity of pain and/or discomfort. In various embodiments, the ear-wearable device can be configured to record detected indicia of pain or other discomfort experienced by the device wearer.

In some embodiments, the system can detect physiological responses to pain or discomfort in order to infer that there is pain or discomfort and/or that there has been a pain event such as changes in heart rate consistent with pain, changes in respiration including respiration rate and/or volume consistent with pain, changes in sympathetic nervous activity consistent with pain, changes in voice pitch and/or sound volume consistent with pain, changes in perspiration consistent with pain, and the like.

Similar to pain, while some degree of stiffness is to be expected after an orthopedic procedure, too much stiffness or an increase in stiffness, may be indicative of a potential problem during the recovery process. As such, in some embodiments herein, can evaluate data to detect and/or infer the occurrence of stiffness and, in some embodiments, a degree of the same. By way of example, in some embodiments, the system herein can use microphone data to detect occurrences of sounds and/or uttered words or phrases (e.g., “I'm so stiff today”) consistent with stiffness. In some embodiments, the system can query the device wearer periodically to detect the occurrence and/or severity of stiffness. In various embodiments, the ear-wearable device can be configured to record detected indicia of stiffness experienced by the device wearer.

In various embodiments, ear-wearable devices herein can be configured to determine a post-procedural mobility value. Similarly, in various embodiments the ear-wearable device can be configured to determine a post-procedural mobility trend. In various embodiments, the ear-wearable device can be configured to determine a post-procedural mobility value and compare the same with a pre-procedure mobility value. To facilitate such a comparison, in various embodiments the ear-wearable device can be configured to determine a mobility baseline value. Mobility and/or mobility values can be determined according to various parameters and/or calculations made using the same. In some embodiments, mobility can be assessed according to one or more of a distance traveled over a time period, a number of steps taken over a time period, an average speed while moving, a distance moved in between rising (such as rising from a chair or a bed) and sitting or lying down events, a speed of rising from a seated or lying posture, an amount of time spent with activity above a threshold level, and the like, and/or a combination of such values. In some embodiments, mobility trends can be determined by comparing mobility values in a given time period versus mobility values in a previous time period. These parameters can be determined using sensor data herein including, but not limited to, motion sensor data, microphone data, geospatial sensor data, heart rate sensor data, and the like. Time periods can be on the order of minutes, hours, days, weeks, or months.

In various embodiments, the ear-wearable device can be configured to evaluate a post-procedural gait parameter of the device wearer. In various embodiments, the ear-wearable device can be configured to evaluate at least one selected from the group consisting of gait velocity, step length, swing and stance phase, double support time, ground reaction forces, impulse, and propulsion during habitual walking. In various embodiments, the ear-wearable device can be configured to detect leg swinging associated with leg stiffness.

In various embodiments, the ear-wearable device can be configured to evaluate a gait parameter symmetry of the device wearer. In various embodiments, the ear-wearable device can be configured to evaluate step pace symmetry of the device wearer. In various embodiments, the ear-wearable device can be configured to detect leaning of the device wearer during ambulation. In various embodiments, the ear-wearable device can be configured to detect stumbling or wobbling in a particular direction. In various embodiments, the ear-wearable device can be configured to detect an asymmetrical leaning pattern of the device wearer during ambulation.

In various embodiments, the ear-wearable device can be configured to detect a posture change or activity of the device wearer and detect a left-right weight distribution during the posture change or activity. In various embodiments, the ear-wearable device can be configured to query the device wearer regarding pain, stiffness, or other discomfort.

Depending on the specific joint being replaced and/or the specific orthopedic procedure that has been performed, various recovery phases are expected to occur. Each phase may include characteristic amounts of pain, physical activity and/or mobility, medications administered, and the like. The system can track observed parameters and/or events against what is expected based on the phase of recovery and/or the elapsed time of recovery to aid in determining whether or not the recovery process is proceeding normally. In some cases, slower than expected advancement of the recovery process (such as slower than expected increases in mobility values) may be indicative of a problem and can be flagged and/or reported on by the system. In some cases, reversals of advancement of the recovery process (such as reduced mobility values after a period of higher mobility values) may be indicative of a problem and can be flagged and/or reported on by the system.

In some cases, too much activity too soon may place an orthopedic patient at risk. For example, undertaking activities that may place too much stress on the joints too soon, or undertaking activities at an intensity that is too high may jeopardize the recovery process and risk setbacks occurring. As such, in some embodiments herein, the system can flag, report on, or otherwise provide information on circumstances where a detected activity level and/or activity type exceeds or is otherwise inconsistent with the recovery phase of the patient. In various embodiments, the ear-wearable device can be configured to provide a notice to the device wearer if a detected post-procedural activity level/value crosses a threshold value.

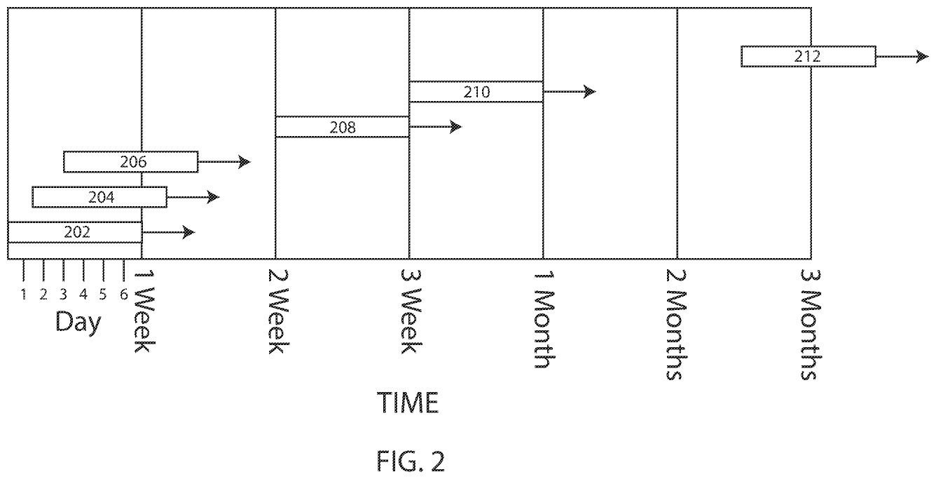

Referring now to FIG. 2, a schematic view of exemplary post-procedural recovery phases is shown in accordance with various embodiments herein. A given exemplary orthopedic procedure can include a first recovery phase 202. As an example, the first recovery phase 202 can begin immediately after the surgery and may include wound care and administration of pain medications with very limited physical activity. In this illustration, the orthopedic procedure also includes a second recovery phase 204. The second recovery phase 204 can begin one to two days after surgery. The second recovery phase 204 can include the initiation of some activity (such as walking with a device such as a walker or a cane) and stretching exercises as directed by a clinician. In this illustration, the orthopedic procedure also includes a third recovery phase 206. The third recovery phase 206 can begin three to four days after surgery and can include increased physical activity along with stretching exercises as directed by a clinician. In this illustration, the orthopedic procedure also includes a fourth recovery phase 208. The fourth recovery phase 208 can begin two to three weeks after surgery and can include activities such as driving along with additional exercise and stretching. In this illustration, the orthopedic procedure also includes a fifth recovery phase 210. The fifth recovery phase 210 can begin three to four weeks after surgery and can include resumption of normal light activities. In this illustration, the orthopedic procedure also includes a sixth recovery phase 212. The sixth recovery phase 212 can begin two to three months after surgery can include the resumption of a broader range of normal activities.

In will be appreciated that the number of recovery phases, the time frames of recovery phases, and the activities associated with recovery phases as described with respect to FIG. 2 are only provided by way of example. In actual operation, embodiments herein can be configured so to work with scenarios having a different number of recovery phases, different time frames of recovery phases, and/or different activities associated with the recovery phases.

In some embodiments, configurations regarding the recovery phases (e.g., the number, time frames, and/or activities of different phases) can be predetermined, such as may be preprogrammed into the system or device. Configurations can also include aspects associated with the recovery phases such as an expected level of mobility or activity and/or range of mobility or activity values. Such configuration values can be predetermined, such as may be preprogrammed into the system or device, or can dynamically determined. In some embodiments, default values for configurations can be used. In some embodiments, templates can be used for such values that are specific to the type of orthopedic procedure (hip replacement, knee replacement, etc.) and/or specific to the type of patient being treated (in terms of age, weight, health status, etc.). In some embodiments, the system or device can be configured to accept input from a clinician, care provider, or other individual regarding recovery phase configurations. In some embodiments, recovery phase configuration information herein can be stored in and/or received from a database.

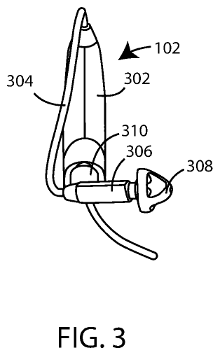

It will be appreciated that ear-wearable devices herein can take on many different specific forms. Referring now to FIG. 3, a schematic view of an exemplary ear-wearable device 102 is shown in accordance with various embodiments herein. The ear-wearable device 102 includes a housing 302 in which various components of the device can be housed. In this example, the ear-wearable device 102 also includes a battery compartment 310. However, some types of ear-wearable devices herein may lack a battery compartment, such as a device with a rechargeable battery. The ear-wearable device 102 also includes a cable 304 which connects to a receiver 306. The ear-wearable device 102 also includes an earbud 308.

It will be appreciated that while FIG. 3 illustrates one type of ear-wearable device (or ear-wearable sensor device) consistent with embodiments herein, many other types of ear-wearable devices are also contemplated. The term “ear-wearable device” as used herein can include devices that can aid a person with impaired hearing. The term “ear-wearable device” shall also refer to devices that can produce optimized or processed sound for persons with normal hearing. Ear-wearable devices herein can include hearing assistance devices. Ear-wearable devices herein can include, but are not limited to, behind-the-ear (BTE), in-the ear (ITE), in-the-canal (ITC), invisible-in-canal (ITC), receiver-in-canal (RIC), receiver in-the-ear (RITE) and completely-in-the-canal (CIC) type hearing assistance devices. In some embodiments, the ear-wearable device can be a hearing aid falling under 21 C.F.R. § 801.420. In some embodiments, the ear-wearable device can be an over-the-counter (OTC) hearing aid. In another example, the ear-wearable device can include one or more Personal Sound Amplification Products (PSAPs). In another example, the ear-wearable device can include one or more cochlear implants, cochlear implant magnets, cochlear implant transducers, and cochlear implant processors. In another example, the hearing assistance device can include one or more “hearable” devices that provide various types of functionality. In other examples, ear-wearable devices can include other types of devices that are wearable in, on, or in the vicinity of the user's ears. In other examples, ear-wearable devices can include other types of devices that are implanted or otherwise osseointegrated with the user's skull; wherein the device is able to facilitate stimulation of the wearer's ears via the bone conduction pathway. In another example, the hearing assistance device can include an auditory brainstem implant, a cranial nerve (e.g., CN VIII) implant, and the like.

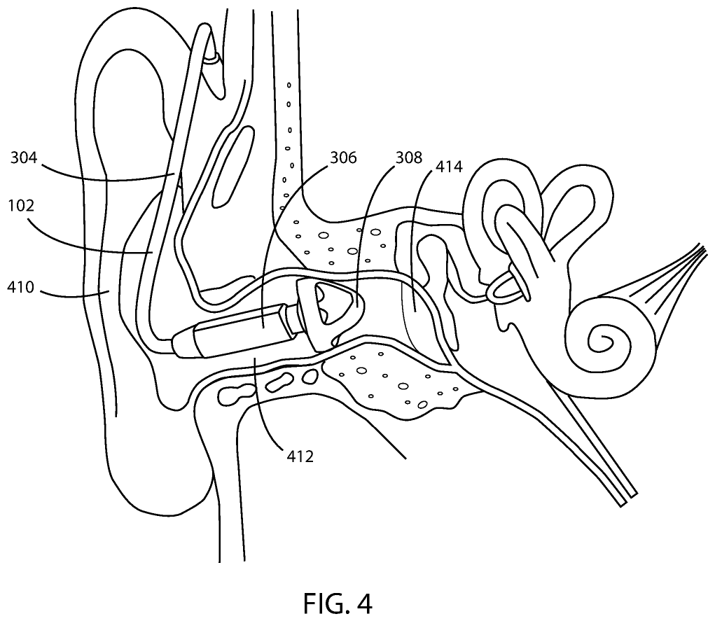

Referring now to FIG. 4, a schematic view of the ear-wearable device 102 is shown with the device fitted in the ear of a device wearer. The significant portions of the ear, in this view, include pinna 410, ear canal 412, and tympanic membrane 414. As before, the ear-wearable device 102 includes a cable 304 connecting to a receiver 306. The ear-wearable device 102 also includes an earbud 308.

In some embodiments herein, such as where two ear-wearable devices are used, the ear-wearable devices can replicate each other's functionality and/or operations such that a check can placed upon identification/detection of orthopedic aspects herein. For example, if one device through its operations determines that a particular orthopedic status is likely present and the other device through its redundant operations does not determine that a particular orthopedic status is likely present then the devices can exchange such data and, to reduce false positives, may not take actions associated with the orthopedic status being detected (such as not sending an alert or other notification). However, if maximum sensitivity is desired at the risk of more false positives, the system can operate in a mode where it determines that a particular orthopedic status is likely present if either of the two devices independently determines that the orthopedic status is likely present. In some embodiments, the devices may not operate redundantly with respect to detection. For example, to conserve battery life, a duty-cycle like process can take place where one device is monitoring during some time periods and the other device is monitoring during other time periods. In some embodiments, the two devices can include the same sensor package and therefore be capable of producing the same types of data. However, in other embodiments, the two devices can include different sensor packages and therefore be capable of producing different types of data.

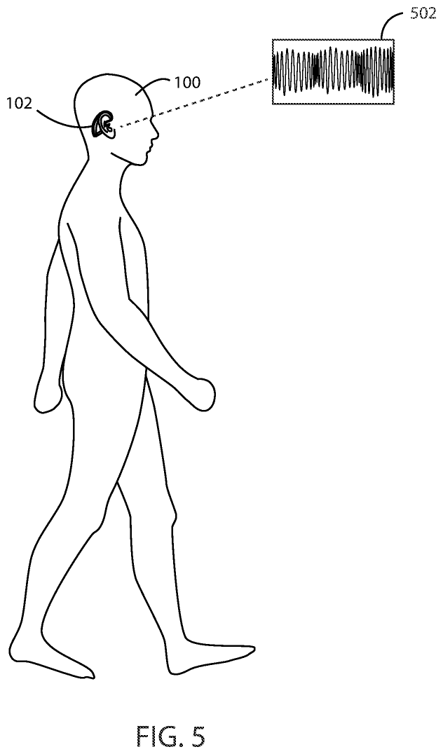

Referring now to FIG. 5, a schematic view of a device wearer 100 with an ear-wearable device 102 is shown in accordance with various embodiments herein. In various embodiments, the sensor package can include a motion sensor, wherein the ear-wearable device 102 can be configured to evaluate aspects of orthopedic status of the device wearer 100 using signals 502 from the motion sensor (motion data) and/or other types of data described herein.

Various aspects related to gait can be used by ear-wearable devices herein to determine orthopedic status. For example, in various embodiments, the ear-wearable device 102 can be configured to evaluate a post-procedural gait parameter of the device wearer 100. In various embodiments, the gait parameter can include at least one of gait velocity, step length, swing and stance phase, double support time, ground reaction forces, impulse, and propulsion while walking. In some embodiments, the ear-wearable device 102 can be configured to detect leg swinging associated with leg stiffness. In some embodiments, the ear-wearable device 102 can be configured to evaluate a gait parameter symmetry of the device wearer 100. In some embodiments, the ear-wearable device 102 can be configured to evaluate step pace symmetry of the device wearer 100. In some embodiments, the ear-wearable device 102 can be configured to detect leaning of the device wearer 100 during ambulation. In some embodiments, the ear-wearable device 102 can be configured to detect stumbling or wobbling in a particular direction. In some embodiments, the ear-wearable device 102 can be configured to detect an asymmetrical leaning pattern of the device wearer 100 during ambulation.

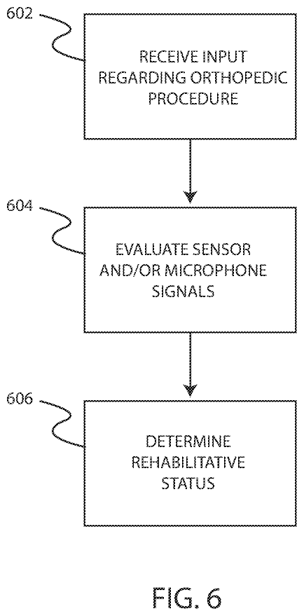

Referring now to FIG. 6, a flow chart is shown of operations of an exemplary embodiment herein. In this example, the operations are performed by the system to monitor an individual after they have had an orthopedic procedure. In a first operation 602, the system can receive an input regarding an occurrence of the orthopedic procedure. In some cases, the input can be provided to the system by a third party such as a care provider or a clinician. In some cases, the input can be provided by the device wearer. In some cases, the input can be provided from another system, such as a medical records system. In alternative embodiments, the system itself can determine that an orthopedic procedure has occurred, such as by detecting the presence of the device wearer at the geolocation of an orthopedic clinician and/or in combination with detecting sensor signal patterns consistent with an orthopedic procedure taking place such as a sudden change in gait. In a second operation 604, the system can evaluate signals from a sensor package and/or a microphone. Various aspects about the device wearer can be determined about by evaluating signals from the sensor package and/or microphone as described elsewhere herein. For example, various gait parameters can be determined regarding the device wearer as described elsewhere herein. In addition, parameters regarding the activity level of the device wearer can be determined as described elsewhere herein. In a third operation 606, the system can determine a rehabilitative status of a device wearer with respect to the orthopedic procedure. For example, the system can determine whether the device wearer is progressing in recovery as they should be. In some embodiments, the system can determine whether activity levels match the recovery stage they should be in based on the amount of time that has elapsed since the orthopedic procedure (see, e.g., FIG. 2 and the associated description). As another example, the system can determine whether or not gait parameters are improving, worsening, or staying the same.

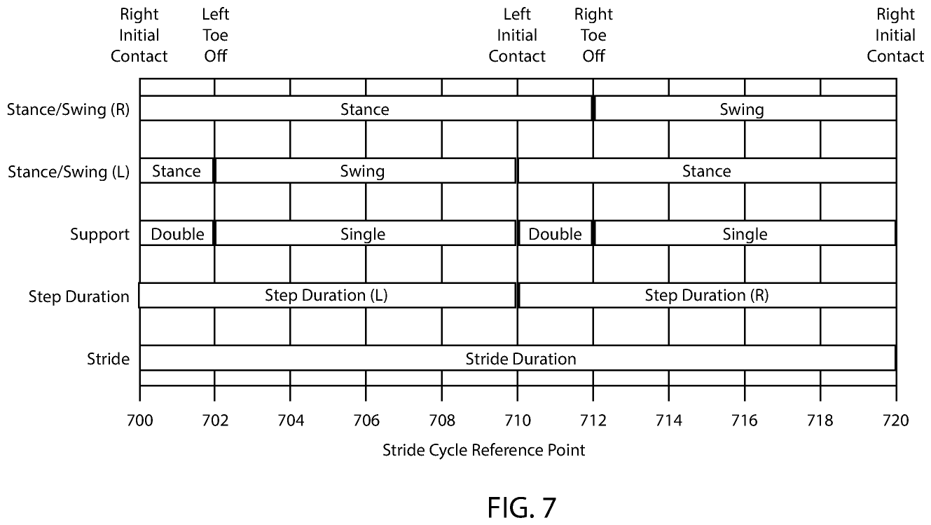

Referring now to FIG. 7, a schematic view of gait aspects is shown in accordance with various embodiments herein. It will be appreciated that a device wearer's stride can be broken down into many different sub-elements for purposes of gait analysis herein. Referring now to FIG. 7, a diagram is shown of events occurring during strides of a device wearer for gait analysis in accordance with various embodiments herein. At reference point 700, the right foot makes initial contact with the ground (foot fall) and both the right leg and the left leg are in a stance. Support is provided by both legs (i.e., double stance) beginning at this time. At reference point 702, the left toe leaves the ground and the left leg enters a swing while the right leg is in a stance. Support is provided by only the right leg (e.g., single) beginning at this time. At reference points 704, 706, and 708 the swing of the left leg continues. At reference point 710, the left foot makes initial contact with the ground and both the right leg and the left leg are in a stance. Support is provided by both legs beginning at this time. At reference point 712, the right toe leaves the ground and the right leg enters a swing while the left leg is in a stance. Support is provided by only the left leg (e.g., single) beginning at this time. At reference points 714, 716, and 718 the swing of the right leg continues. Reference point 720 marks the conclusion of the stride cycle whereupon if the device wearer continues to walk the cycle will repeat beginning at reference point 700.

In accordance with embodiments herein, one or more of a motion sensor and a microphone herein can detect movements and/or vibrations in order to identify what stage of the stride cycle the device wearer is currently in along with frequencies and time associated with the same. By way of example, reference points 700 and 710 involve the right and left feet, respectively, making initial contact with the ground. The biomechanics associated with such feet/ground contact results in characteristic acoustic and inertial changes that can be detected by one or more microphones and/or accelerometers (or other component) of a motion sensor, either alone or in combination. In some embodiments, characteristics of feet/ground contact can include a signal intensity. In some embodiments, characteristics of feet/ground contact can include a time interval. In some embodiments the spectral intensity and timing of a first, second, third, etc. microphone may be compared, summed, or subtracted to determine the spatial location of a foot fall. In some embodiments, the system may determine if the footfalls are associated with the wearer or if the footfalls are associated with another individual. In some embodiments, the system may also determine if it is the left foot or the right foot making the footfall. In some embodiments, characteristics of feet/ground contact can include an angular position of one or more parts of the body. For example, as one leg swings forward (e.g., starting at reference point 702 and ending at reference point 710 for the left leg and starting at reference point 712 and ending at reference point 720 for the right leg) support by the other leg involves a characteristic vertical motion at a relatively low frequency that can be detected by a component of the motion sensor.

In some embodiments herein, a heart rate or PPG sensor herein can detect and/or confirm detection of footfalls. A footfall can generate a detectable signal using a heart rate or PPG sensor. In some embodiments, magnitude of motion sensor signals along with heart rate values can be used to differentiate between shuffling, typical walking, and movement due to an external force. This is because the signal from a heart rate or PPG sensor will vary depending on whether the device wearer is shuffling, exhibiting typical walking, or undergoing other movement. In some embodiments, the detection of footfalls with a motion sensor and also with a heart rate or PPG sensor can provide confirmation that the device wearer is actually wearing the ear-wearable devices as intended and not just storing them in their pockets. This is because the devices may still register footfalls with a motion sensor even if the ear-wearable device are not being worn, but a heart rate or PPG sensor associated with the device would not provide a useful signal if the device is not being worn.

Characteristic medio-lateral axis movement can also be detected by the motion sensor during different phases of the stride cycle allowing each point to be identified along with timing of the same. Left versus right steps can also be distinguished by evaluating detected medio-lateral axis movement. By way of example, a limping gait can be reflected as unequal swing durations between each leg and this type of abnormal or atypical gait can be detected by the system. As another example, a shuffling-type gait can be reflected as a measurable variability in the timing of the different phases of the stride cycle that crosses a threshold value of variability or statistics (the threshold value either being pre-selected and programmed into the device or reflecting a statistical measure of deviation from another statistical measure, e.g., an average, for the specific individual as calculated over a look-back period or during a previous calibration period or event). A shuffling-type gait or other scenarios can also be detected using acoustic information obtained from one or more microphones.

In addition, by combining the information content provided by signals associated with directional movement in the horizontal plane (as can be measured by the motion sensor, microphone, or geolocation-type sensors) with that provided by stride cycle analysis as detailed above, aspects such as step length (right, left) and stride length can be calculated. These values can also be subjected to analysis to determine various statistics, e.g., absolute values (average right step length, average left step length, average stride length) as well as ratios of the same (ratio of average right step length vs. average left step length) and measures of variability or other statistics in the same, and the like.

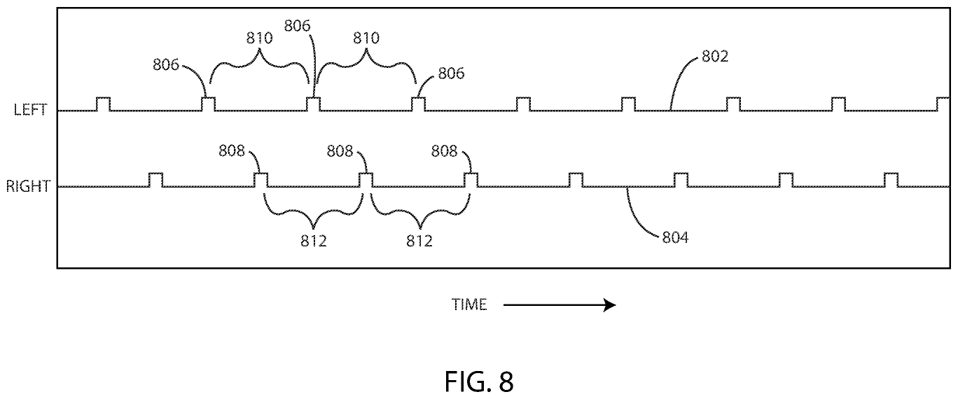

Referring now to FIG. 8, a schematic view of left and right steps is shown in accordance with various embodiments herein. This view shows left side gait activity 802 including left-side steps 806. The left side gait activity 802 includes a left step interval 810 between successive left-side steps 806. This view also shows right side gait activity 804 including right side step 808. The right side gait activity 804 includes a right step interval 812 between successive right-side steps 808. As previously discussed, in various embodiments the ear-wearable device can be configured to record signals from at least one sensor, such as at least one of the motion sensor and the microphone, and process the signals to characterize an existing gait of the device wearer, including left side gait activity 802 and right side gait activity 804 in order to determine an orthopedic status of the device wearer.

In some cases, the timing of detecting orthopedic related aspects and/or things such as assessing pain, stiffness, or other discomfort can be significant. For example, aspects such as pain experienced while rising from a chair or during another change in posture may be more or less significant as an indicator of the recovery process than is an amount of pain experienced while steadily walking. Certain changes in posture and certain movements can place more stress on the joints thus making the occurrence of pain, stiffness, or other discomfort more likely when a patient is changing their posture or undertaking certain movements. This can be important to consider when determining the significance in a change of a pain, stiffness, or discomfort level. For example, if an increase in pain occurs at the same time as rising from a chair, then this may be expected.

However, if an increase in pain occurs in the absence of a postural change or specific movement then this may be significant. As such, in some embodiments, motion sensor data and/or microphone data gathered as described herein is evaluated along with data relevant for detecting and/or monitoring pain, stiffness, and/or other discomfort. In some embodiments, postural change is evaluated in combination with data relevant for detecting and/or monitoring pain, stiffness, and/or other discomfort. In various embodiments, the ear-wearable device can be configured to change a pain, stiffness, or other discomfort detection threshold value when the device wearer is detected to be sitting down, standing up, using stairs, or exercising.

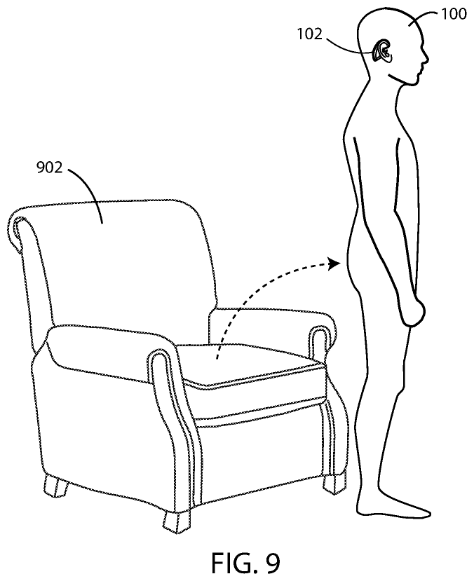

Referring now to FIG. 9, a schematic view of a device wearer 100 rising from a chair 902 is shown in accordance with various embodiments herein. FIG. 9 shows an ear-wearable device 102. In various embodiments, the ear-wearable device 102 can be configured to detect a posture change or activity of the device wearer 100. In various embodiments, the ear-wearable device 102 can be configured to change a pain, stiffness, or other discomfort detection threshold value when the device wearer 100 can be detected to be sitting down, standing up, using stairs, or exercising. In various embodiments, the ear-wearable device 102 can be configured to detect a posture change or activity of the device wearer 100 and detect a left-right weight distribution during the posture change or activity.

In some embodiments, the ear-wearable device and/or system herein can also detect posture associated with gait. For example, if the device wearer is leaning too far (forward, backward, or to the side) while walking, this may negatively impact gait as well as generate an elevated fall risk. Further, the detected posture can be relevant for determining the device wearer's orthopedic status. Posture can be detected using various sensors herein including, for example, an accelerometer that may be part of a motion sensor herein. In some embodiments, sensors herein can also include a gyroscope that can be used to detect angular deviations associated with gait, such as leaning forward.

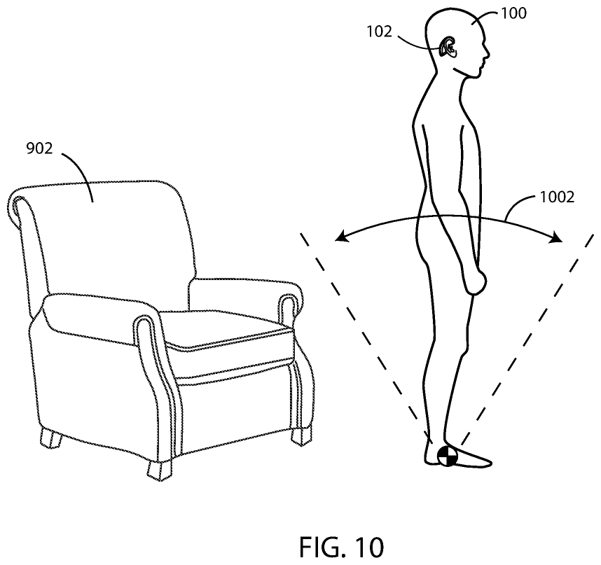

Referring now to FIG. 10, a schematic view of a device wearer 100 getting up from a chair 902 is shown in accordance with various embodiments herein. FIG. 10 also shows the device wearer 100 with an ear-wearable device 102. The device wearer 100 is illustrated exhibiting a degree of sway 1002 (front to back, side to side, etc.). Sway 1002 can be measured using signals from a motion sensor. The magnitude of sway 1002 can be evaluated by the ear-wearable device 102.

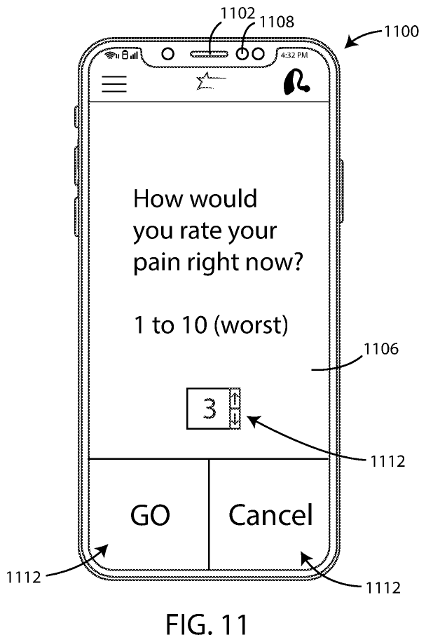

In various embodiments, devices herein can interface with an accessory device and/or systems herein can include an accessory device. Referring now to FIG. 11, a schematic view of an accessory device 1100 is shown in accordance with various embodiments herein. The accessory device 1100 can include a display screen 1106 thereof. The accessory device 1100 can also include a speaker 1102 and a front-facing camera 1108.

In various embodiments, the ear-wearable device can be configured to query the device wearer 100 regarding pain, stiffness, or other discomfort. In some cases, this can be done by directly by the ear-wearable device and in some cases this can be done utilizing the accessory device 1100. For example, a query can be displayed using the display screen 1106 and the device wearer and provide input using one or more input elements 1112.

In many cases, a clinician or other care provider may want the device wearer to undertake certain activities (exercises, stretching, and the like) as part of the recovery process from an orthopedic procedure. In various embodiments, the ear-wearable device can be configured to provide reminders to the device wearer regarding recommended post-procedural activities. In various embodiments, the recommended post-procedural activities can include stretching and/or exercises. Similarly, in some cases the clinician or other care provider may want the device wearer to avoid certain activities and/or avoid a certain intensity or duration of activities. For example, if the ear-wearable device detects that the device wearer is running by identifying a motion sensor and/or microphone signal pattern consistent with running, it could provide the device wearer with a reminder that they shouldn't be running or should limit their run to no more than 15 minutes. The device can also notify the care provider or another third party when instances of activity to avoid are detected. In some embodiments, the device can provide suggestions on alternatives, such as an elliptical machine or biking versus running. Similarly, in some cases the clinician or other care provider may want the device wearer to avoid an amount or magnitude of impact force crossing a threshold value and/or a total amount of aggregated impacts crossing a threshold value. As such, the ear-wearable device can track impacts (using motion sensor data) and/or record information regarding the same. The ear-wearable-device can send a notice or reminder to the device wearer and/or a care provider if sensed impacts exceed a threshold value of magnitude and/or if a total amount of sensed impacts exceeds a threshold value for an aggregate amount of impact exposure. Threshold values can be input by a system user, the device wearer, and/or can be received from a separate system. In some cases, threshold values can be set based on the type of orthopedic procedure the device wearer has experienced. For example, a lookup table can be consulted to provide default impact threshold values based on the type of orthopedic procedure. In some embodiments, the device can provide suggestions on lower impact alternative activities, such as an elliptical machine or biking versus running.

In some cases the reminders, instructions, and/or suggestions can be provided directly and in some cases they can be provided via an accessory device. Reminders, instructions, and/or suggestions can be provided audibly, visually, and or haptically.

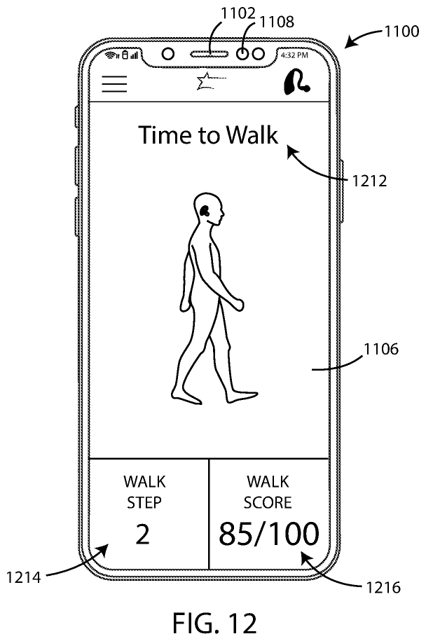

Referring now to FIG. 12, a schematic view of an accessory device 1100 is shown in accordance with various embodiments herein. As before, the accessory device 1100 can include a display screen 1106 thereof. The accessory device 1100 can also include a speaker 1102 and a front-facing camera 1108. In this case, an instruction 1212 or reminder can be provided via the accessory device 1100 and various information regarding the same 1214, 1216 can also be provided to the device wearer via the accessory device 1100.

In some cases, infection can be a risk during recovery from an orthopedic procedure. In various embodiments, the ear-wearable device can be configured to evaluate signals from a sensor package to detect possible post-procedural infection. For example, ear-wearable devices configured to generate a set of data reflecting current physiological parameters and/or related data of a device wearer based on signals from sensors and/or data received from other devices and match the set of data against a plurality of predetermined patterns (which can include positive example patterns, negative example patterns, and the like) to determine whether the device wearer currently has an infection. In various embodiments, the ear-wearable device can be configured to send an alert to a care provider if a possible post-procedural infection is detected. The data used for pattern matching can include one or more of temperature data, motion sensor data, sound data, cardiac data, temperature data, respiration data, blood pressure data, oxygen saturation (SpO2) data, and the like, or combinations of these. In some embodiments, ear-wearable devices herein can be configured to generate a set of data reflecting a current health status of a device wearer based on signals from one or more sensors and compare the set of data against stored data reflecting a previous health status of the device wearer and characterize an infection status of the device wearer based on a change from the previous health status to the current health status of the device wearer. Aspects regarding exemplary infection detection techniques are described in U.S. patent application Ser. No. 17/834,569, the content of which is herein incorporated by reference.

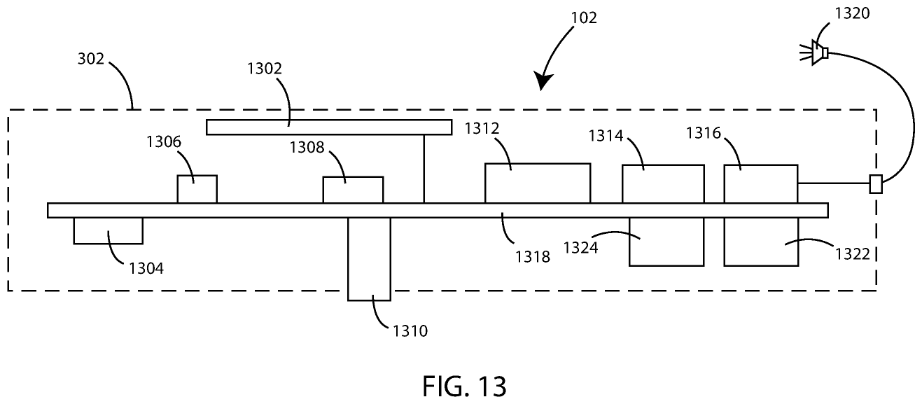

Ear-wearable devices herein can include many different components. Referring now to FIG. 13, a schematic block diagram is shown illustrating various components of an ear-wearable device in accordance with various embodiments herein. It will be appreciated that many of these components can be integrated in an integrated circuit, such as with a system-on-a-chip (SOC) integration or can exist as separate components. The block diagram of FIG. 13 represents a generic ear-wearable device for purposes of illustration. The ear-wearable device 102 shown in FIG. 13 includes several components electrically connected to a flexible mother circuit 1318 (e.g., flexible mother board) which is disposed within housing 302. A power supply circuit 1304 can include a battery and can be electrically connected to the flexible mother circuit 1318 and provides power to the various components of the ear-wearable device 102. One or more microphones 1306 are electrically connected to the flexible mother circuit 1318, which provides electrical communication between the microphones 1306 and a digital signal processor (DSP) 1312. Among other components, the DSP 1312 incorporates or is coupled to audio signal processing circuitry configured to implement various functions described herein. A sensor package 1314 can be coupled to the DSP 1312 via the flexible mother circuit 1318. The sensor package 1314 can include one or more different specific types of sensors such as those described in greater detail below. One or more user switches 1310 (e.g., on/off, volume, mic directional settings) are electrically coupled to the DSP 1312 via the flexible mother circuit 1318.

An audio output device 1316 is electrically connected to the DSP 1312 via the flexible mother circuit 1318. In some embodiments, the audio output device 1316 comprises a speaker (coupled to an amplifier). In other embodiments, the audio output device 1316 comprises an amplifier coupled to an external receiver 1320 adapted for positioning within an ear of a wearer. The external receiver 1320 can include an electroacoustic transducer, speaker, or loudspeaker. The ear-wearable device 102 may incorporate a communication device 1308 coupled to the flexible mother circuit 1318 and to an antenna 1302 directly or indirectly via the flexible mother circuit 1318. The communication device 1308 can be a Bluetooth® transceiver, such as a BLE (Bluetooth® low energy) transceiver or other transceiver(s) (e.g., an IEEE 802.11 compliant device). The communication device 1308 can be configured to communicate with one or more external devices, such as those discussed previously, in accordance with various embodiments. In various embodiments, the communication device 1308 can be configured to communicate with an external visual display device such as a smart phone, a video display screen, a tablet, a computer, a television, a virtual or augmented reality, a hologram, or the like.

In various embodiments, the ear-wearable device 102 can also include a control circuit 1322 and a memory storage device 1324. The control circuit 1322 can be in electrical communication with other components of the device. The control circuit 1322 can execute various operations, such as those described herein. The control circuit 1322 can include various components including, but not limited to, a microprocessor, a microcontroller, an FPGA (field-programmable gate array) processing device, an ASIC (application specific integrated circuit), or the like. The memory storage device 1324 can include both volatile and non-volatile memory. The memory storage device 1324 can include ROM, RAM, flash memory, EEPROM, SSD devices, NAND chips, and the like. The memory storage device 1324 can be used to store data from sensors as described herein and/or processed data generated using data from sensors as described herein.

In various embodiments, a spatial location determining circuit (or geolocation circuit) can be included and can take the form of an integrated circuit that can include components for receiving signals from GPS, GLONASS, BeiDou, Galileo, SBAS, WLAN, BT, FM, and/or NFC type protocols.

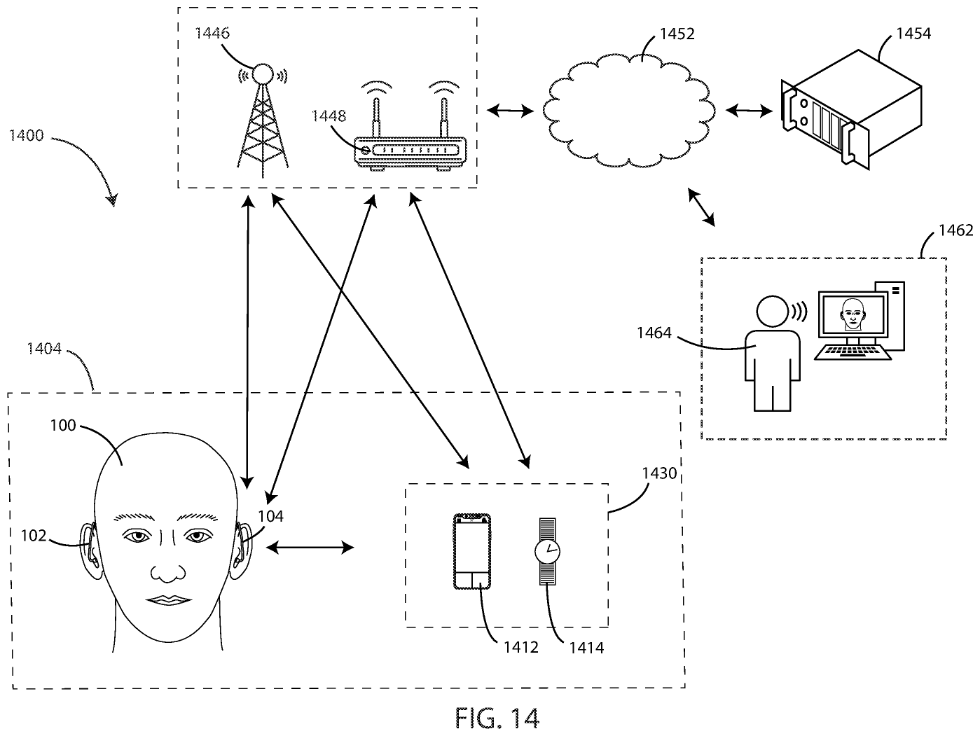

It will be appreciated that systems herein can include and/or interface with many different devices or components. Referring now to FIG. 14, a schematic view of various components of a system 1400 is shown in accordance with various embodiments herein. A local environment 1404 can include the device wearer 100 along with an ear-wearable device 102 and a second ear-wearable device 104. Various accessory devices 1430 can also be associated with the device wearer 100 and/or can contain or gather data regarding the device wearer 100 in the local environment 1404. The accessory devices 1430 can include, but are not limited to, a smart phone 1412 and a wearable device 1414, amongst others. Various data communication elements can be used to convey data transmissions to or from device and/or system herein. By way of example, FIG. 14 shows a cell tower 1446 and a router 1448, each of which can be used to convey data communications to or from devices and/or system components herein. In some embodiments, data communications can be conveyed to or from the cloud 1452. The cloud 1452 can include various resources such as a remote server 1454 (which could be real or virtual) as well as other processing and/or data storage devices (real or virtual). In some embodiments, processing steps herein of data can be performed in the cloud 1452. In some embodiments, data can be retrieved from the cloud 1452 (and/or a medical record system that can be in or communicate through the cloud 1452 or another data network) and provided to devices and/or systems herein such as medical record data to allow access to health information regarding the device wearer. In various embodiments, the ear-wearable device 102 can specifically be configured to receive information regarding previous infections of a device wearer 100.

FIG. 14 also shows a remote environment 1462. The remote environment 1462 can include a third party 1464. The third party 1464 can be a recipient of information and/or alerts sent in accordance with embodiments herein. The third party 1464 can be a care provider, a clinician such as an orthopedic specialist, a family member, or the like. In various embodiments, the ear-wearable device 102 can be configured to change a threshold for detecting the risk of the presence of an infection based on an input from a third party 1464, such as a care provider or a clinician.

Accessory devices (or secondary devices) can be useful sources of data in accordance with embodiments herein. By way of example, accessory devices, such as wearable devices can gather cardiac data, respiration data, blood pressure data, sound data, temperature data, SpO2 data, and the like. In various embodiments, the ear-wearable device 102 can be configured to receive data regarding physiological parameters from a separate wearable device 1414.

### Methods

Many different methods are contemplated herein, including, but not limited to, methods of making, methods of using, and the like. Aspects of system/device operation described elsewhere herein can be performed as operations of one or more methods in accordance with various embodiments herein.

In various embodiments, operations described herein and method steps can be performed as part of a computer-implemented method executed by one or more processors of one or more computing devices. In various embodiments, operations described herein and method steps can be implemented instructions stored on a non-transitory, computer-readable medium that, when executed by one or more processors, cause a system to execute the operations and/or steps.

In an embodiment, a method of monitoring an individual after an orthopedic procedure is included. The method can include receiving an input regarding an occurrence of the orthopedic procedure, evaluating signals from a sensor package and/or a microphone, and determining a rehabilitative status of a device wearer with respect to the orthopedic procedure.

In an embodiment, the method can further include determining a post-procedural mobility value. In an embodiment, the method can further include determining a post-procedural mobility trend.

In an embodiment, the method can further include determining a post-procedural mobility value and comparing the same with a pre-procedure mobility value.

In an embodiment, the method can further include evaluating a post-procedural gait parameter of the device wearer. In an embodiment, the method can further include detecting leg swinging associated with leg stiffness. In an embodiment, the method can further include evaluating a gait parameter symmetry of the device wearer. In an embodiment, the method can further include evaluating step pace symmetry of the device wearer. In an embodiment, the method can further include detecting leaning of the device wearer during ambulation. In an embodiment, the method can further include detecting stumbling or wobbling in a particular direction. In an embodiment, the method can further include detecting an asymmetrical leaning pattern of the device wearer during ambulation.

In an embodiment, the method can further include evaluating signals from the sensor package and/or the microphone for indicia of pain. In an embodiment, the method can further include evaluating signals from the microphone to detect one or more phrases or utterances indicative of pain, stiffness, or other discomfort associated with the orthopedic procedure.

In an embodiment, the method can further include detecting a posture change or activity of the device wearer. In an embodiment, the method can further include changing a pain, stiffness, or other discomfort detection threshold value when the device wearer is detected to be sitting down, standing up, using stairs, or exercising. In an embodiment, the method can further include detecting a posture change or activity of the device wearer and detecting a left-right weight distribution during the posture change or activity.

In an embodiment, the method can further include querying the device wearer regarding pain, stiffness, or other discomfort. In an embodiment, the method can further include recording detected indicia of pain, stiffness, or other discomfort experienced by the device wearer.

In an embodiment, the method can further include evaluating signals from the sensor package to detect possible post-procedural infection. In an embodiment, the method can further include sending an alert to a care provider if a possible post-procedural infection is detected.

In an embodiment, the method can further include providing information to a care provider regarding a rehabilitative status of the device wearer. In an embodiment, the method can further include providing a notice to the device wearer if a detected post-procedural activity level crosses a threshold value.

In an embodiment, the method can further include providing reminders to the device wearer regarding recommended post-procedural activities. In an embodiment, the method can further include providing instructions to the device wearer regarding recommended post-procedural activities.

### Pattern Identification and Pattern Matching

It will be appreciated that in various embodiments herein, a device or a system can detect an orthopedic status of a device wearer by evaluating data and identifying a pattern regarding the same and/or matching the data against patterns or templates that are indicative of specific orthopedic statuses. Patterns can be identified and/or matched in various ways. As merely one example, one or more sensors can be operatively connected to a controller (such as the control circuit describe in FIG. 13) or another processing resource (such as a processor of another device or a processing resource in the cloud).

The controller or other processing resource can be adapted to receive data representative of a characteristic of the subject from one or more of the sensors and/or determine statistics of the subject over a monitoring time period based upon the data received from the sensor. As used herein, the term “data” can include a single datum or a plurality of data values or statistics. The term “statistics” can include any appropriate mathematical calculation or metric relative to data interpretation, e.g., probability, confidence interval, distribution, range, or the like. Further, as used herein, the term “monitoring time period” means a period of time over which characteristics of the subject are measured and statistics are determined. The monitoring time period can be any suitable length of time, e.g., 1 millisecond, 1 second, 10 seconds, 30 seconds, 1 minute, 10 minutes, 30 minutes, 1 hour, etc., or a range of time between any of the foregoing time periods.

Any suitable technique or techniques can be utilized to determine statistics for the various data from the sensors, e.g., direct statistical analyses of time series data from the sensors, differential statistics, comparisons to baseline or statistical models of similar data, etc. Such techniques can be general or individual-specific and represent long-term or short-term behavior. These techniques could include standard pattern classification methods such as Gaussian mixture models, clustering as well as Bayesian approaches, neural network models and deep learning.

Further, in some embodiments, the controller can be adapted to compare data, data features, and/or statistics against various other patterns, which could be prerecorded patterns (baseline patterns) of the particular individual wearing an ear-wearable device herein, prerecorded patterns (group baseline patterns) of a group of individuals wearing ear-wearable devices herein, one or more predetermined patterns that serve as patterns indicative of specific orthopedic statuses (positive example patterns), one or more predetermined patterns that service as patterns indicative of the absence of specific orthopedic statuses (negative example patterns), or the like. As merely one scenario, if a pattern is detected in an individual that exhibits similarity crossing a threshold value to a positive example pattern or substantial similarity to that pattern, then that can be taken as an indication of the individual having the orthopedic status associated with the example pattern.

Similarity and dissimilarity can be measured directly via standard statistical metrics such normalized Z-score, or similar multidimensional distance measures (e.g. Mahalanobis or Bhattacharyya distance metrics), or through similarities of modeled data and machine learning. These techniques can include standard pattern classification methods such as Gaussian mixture models, clustering as well as Bayesian approaches, neural network models, and deep learning.

As used herein the term “substantially similar” means that, upon comparison, the sensor data are congruent or have statistics fitting the same statistical model, each with an acceptable degree of confidence. The threshold for the acceptability of a confidence statistic may vary depending upon the subject, sensor, sensor arrangement, type of data, context, condition, etc.

The statistics associated with the health status of an individual (and, in particular, their status with respect to an orthopedic status), over the monitoring time period, can be determined by utilizing any suitable technique or techniques, e.g., standard pattern classification methods such as Gaussian mixture models, clustering, hidden Markov models, as well as Bayesian approaches, neural network models, and deep learning.

### Sensors

Ear-wearable devices as well as medical devices herein can include one or more sensor packages (including one or more discrete or integrated sensors) to provide data. The sensor package can comprise one or a multiplicity of sensors. In some embodiments, the sensor packages can include one or more motion sensors (or movement sensors) amongst other types of sensors. Motion sensors herein can include inertial measurement units (IMU), accelerometers, gyroscopes, barometers, altimeters, and the like. The IMU can be of a type disclosed in commonly owned U.S. Pat. No. 9,848,273, filed Oct. 21, 2016, which is incorporated herein by reference. In some embodiments, electromagnetic communication radios or electromagnetic field sensors (e.g., telecoil, NFMI, TMR, GMR, etc.) sensors may be used to detect motion or changes in position. In some embodiments, biometric sensors may be used to detect body motions or physical activity. Motions sensors can be used to track movement of a patient in accordance with various embodiments herein.

In some embodiments, the motion sensors can be disposed in a fixed position with respect to the head of a patient, such as worn on or near the head or ears. In some embodiments, the operatively connected motion sensors can be worn on or near another part of the body such as on a wrist, arm, or leg of the patient.

According to various embodiments, the sensor package can include one or more of an IMU, and accelerometer (3, 6, or 9 axis), a gyroscope, a barometer, an altimeter, a magnetometer, a magnetic sensor, an eye movement sensor, a pressure sensor, an acoustic sensor, a telecoil, a heart rate sensor, a global positioning system (GPS), a temperature sensor, a blood pressure sensor, an oxygen saturation sensor, an optical sensor, a blood glucose sensor (optical or otherwise), a galvanic skin response sensor, a cortisol level sensor (optical or otherwise), a microphone, acoustic sensor, an electrocardiogram (ECG) sensor, electroencephalography (EEG) sensor which can be a neurological sensor, eye movement sensor (e.g., electrooculogram (EOG) sensor), myographic potential electrode sensor (EMG), a heart rate monitor, a pulse oximeter or oxygen saturation sensor (SpO2), a wireless radio antenna, blood perfusion sensor, hydrometer, sweat sensor, cerumen sensor, air quality sensor, pupillometry sensor, cortisol level sensor, hematocrit sensor, light sensor, image sensor, and the like.

In some embodiments, spatial location sensors and/or geolocation sensors can be included and can take the form of an integrated circuit that can include components for receiving signals from GPS, GLONASS, BeiDou, Galileo, SBAS, WLAN, BT, FM, and/or NFC type protocols

In some embodiments, the sensor package can be part of an ear-wearable device. However, in some embodiments, the sensor packages can include one or more additional sensors that are external to an ear-wearable device. For example, various of the sensors described above can be part of a wrist-worn or ankle-worn sensor package, or a sensor package supported by a chest strap. In some embodiments, sensors herein can be disposable sensors that are adhered to the device wearer (“adhesive sensors”) and that provide data to the ear-wearable device or another component of the system.

Data produced by the sensor(s) of the sensor package can be operated on by a processor of the device or system.

As used herein the term “inertial measurement unit” or “IMU” shall refer to an electronic device that can generate signals related to a body's specific force and/or angular rate. IMUs herein can include one or more accelerometers (3, 6, or 9 axis) to detect linear acceleration and a gyroscope to detect rotational rate. In some embodiments, an IMU can also include a magnetometer to detect a magnetic field.

The eye movement sensor may be, for example, an electrooculographic (EOG) sensor, such as an EOG sensor disclosed in commonly owned U.S. Pat. No. 9,167,356, which is incorporated herein by reference. The pressure sensor can be, for example, a MEMS-based pressure sensor, a piezo-resistive pressure sensor, a flexion sensor, a strain sensor, a diaphragm-type sensor and the like.

The temperature sensor can be, for example, a thermistor (thermally sensitive resistor), a resistance temperature detector, a thermocouple, a semiconductor-based sensor, an infrared sensor, or the like.

The blood pressure sensor can be, for example, a pressure sensor. The heart rate sensor can be, for example, an electrical signal sensor, an acoustic sensor, a pressure sensor, an infrared sensor, an optical sensor, or the like.

The oxygen saturation sensor (such as a blood oximetry sensor) can be, for example, an optical sensor, an infrared sensor, a visible light sensor, or the like.

The electrical signal sensor can include two or more electrodes and can include circuitry to sense and record electrical signals including sensed electrical potentials and the magnitude thereof (according to Ohm's law where V=IR) as well as measure impedance from an applied electrical potential.

It will be appreciated that the sensor package can include one or more sensors that are external to the ear-wearable device. In addition to the external sensors discussed hereinabove, the sensor package can comprise a network of body sensors (such as those listed above) that sense movement of a multiplicity of body parts (e.g., arms, legs, torso). In some embodiments, the ear-wearable device can be in electronic communication with the sensors or processor of another medical device, e.g., an insulin pump device or a heart pacemaker device.

It should be noted that, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

As used herein, the recitation of numerical ranges by endpoints shall include all numbers subsumed within that range (e.g., 2 to 8 includes 2.1, 2.8, 5.3, 7, etc.).

The headings used herein are provided for consistency with suggestions under 37 CFR 1.77 or otherwise to provide organizational cues. These headings shall not be viewed to limit or characterize the invention(s) set out in any claims that may issue from this disclosure. As an example, although the headings refer to a “Field,” such claims should not be limited by the language chosen under this heading to describe the so-called technical field. Further, a description of a technology in the “Background” is not an admission that technology is prior art to any invention(s) in this disclosure. Neither is the “Summary” to be considered as a characterization of the invention(s) set forth in issued claims.

The embodiments described herein are not intended to be exhaustive or to limit the invention to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

1. An ear-wearable device comprising:
a control circuit;
a microphone, wherein the microphone is in electrical communication with the control circuit; and
a sensor package, wherein the sensor package is in electrical communication with the control circuit;
wherein the ear-wearable device is configured to
receive an input regarding an occurrence of an orthopedic procedure;
evaluate signals from the sensor package and/or the microphone; and
determine a rehabilitative status of a device wearer with respect to the orthopedic procedure.

2-4. (canceled)

5. The ear-wearable device of claim 1, wherein the ear-wearable device is configured to evaluate signals from the sensor package and/or the microphone for indicia of pain.

6-7. (canceled)

8. The ear-wearable device of claim 1, wherein the ear-wearable device is configured to determine a post-procedural mobility value and compare the same with a pre-procedure mobility value.

9. The ear-wearable device of claim 1, wherein the ear-wearable device is configured to evaluate a post-procedural gait parameter of the device wearer.

10. The ear-wearable device of claim 9, the gait parameter comprising at least one selected from the group consisting of gait velocity, step length, swing and stance phase, double support time, ground reaction forces, impulse, and propulsion during habitual walking.

11. The ear-wearable device of claim 1, wherein the ear-wearable device is configured to detect leg swinging associated with leg stiffness.

12. The ear-wearable device of claim 1, wherein the ear-wearable device is configured to evaluate a gait parameter symmetry of the device wearer.

13-15. (canceled)

16. The ear-wearable device of claim 1, wherein the ear-wearable device is configured to detect an asymmetrical leaning pattern of the device wearer during ambulation.

17. The ear-wearable device of claim 1, wherein the ear-wearable device is configured to evaluate signals from the microphone to detect one or more phrases or utterances indicative of pain, stiffness, or other discomfort associated with the orthopedic procedure.

18. The ear-wearable device of claim 17, wherein the ear-wearable device is configured to detect a posture change or activity of the device wearer.

19. The ear-wearable device of claim 17, wherein the ear-wearable device is configured to change a pain, stiffness, or other discomfort detection threshold value when the device wearer is detected to be sitting down, standing up, using stairs, or exercising.

20. The ear-wearable device of claim 1, wherein the ear-wearable device is configured to detect a posture change or activity of the device wearer and detect a left-right weight distribution during the posture change or activity.

21. The ear-wearable device of claim 1, wherein the ear-wearable device is configured to query the device wearer regarding pain, stiffness, or other discomfort.

22. (canceled)

23. The ear-wearable device of claim 1, wherein the ear-wearable device is configured to evaluate signals from the sensor package to detect possible post-procedural infection.

24. The ear-wearable device of claim 23, wherein the ear-wearable device is configured to send an alert to a care provider if a possible post-procedural infection is detected.

25-28. (canceled)

29. The ear-wearable device of claim 1, wherein the ear-wearable device is configured to provide a reminder to the device wearer if a duration of a detected post-procedural activity crosses a threshold value.

30. The ear-wearable device of claim 1, wherein the ear-wearable device is configured to provide a reminder to the device wearer if a post-procedural activity that is not recommended is detected.

31. The ear-wearable device of claim 1, wherein the ear-wearable device is configured to provide reminders to the device wearer regarding recommended post-procedural activities.

32. The ear-wearable device of claim 31, the recommended post-procedural activities comprising stretching and/or exercises.

33. The ear-wearable device of claim 1, wherein the ear-wearable device is configured to provide instructions to the device wearer regarding recommended post-procedural activities.

34-80. (canceled)
